(19) United States
(12) Patent Application Publication
(10) Pub. No.: US 2012/0089183 A1
(43) Pub. Date: Apr. 12, 2012

(54) SPINAL TRACTION DEVICE

(75) Inventors: Mark L. Gostine, Grand Rapids, MI (US); Todd J. Staniulis, Comstock Park, MI (US)

(73) Assignee: Mark L. Gostine, Grand Rapids, MI (US)

(21) Appl. No.: 13/325,601

(22) Filed: Dec. 14, 2011

Related U.S. Application Data
(60) Provisional application No. 61/428,512, filed on Dec. 30, 2010.

(51) Int. Cl. A61F 5/00

(52) U.S. Cl. 606/240

(57) ABSTRACT

A spinal traction device permits a user to conduct spinal traction therapies, either with or without aid by another, using the user's leg weight and/or leg muscle and/or torso muscle action to apply tensile forces to the spine. The device includes a body support portion on which the user lies in a supine position, and a movable leg support that is coupled to the body support portion. The leg support pivots and/or translates relative to the body support portion in response to the application of leg weight and/or leg muscle and/or torso muscle action to the leg support. Under-arm supports along the body support portion engage the user's underarms to keep the user's upper torso in a substantially fixed position, so that tension is applied to the user's spine in response to movement of the leg support, as it is engaged by the calves and/or backs of the user's legs.

### CROSS REFERENCE TO RELATED APPLICATION

The present application claims the benefit of U.S. provisional application Ser. No. 61/428,512, filed Dec. 30, 2010, which is hereby incorporated herein by reference in its entirety.

### FIELD OF THE INVENTION

The present invention is directed to traction devices, and more particularly, to traction devices for conducting spinal therapies.

### BACKGROUND OF THE INVENTION

Spinal traction is generally used to stretch the spine and/or muscles associated with the spine, to reduce back pain, straighten or align the spine, or treat various spine conditions. Various devices have been developed to perform spinal traction therapies, ranging from stretching tables using mechanically-operated stretching devices, to inversion tables that use a user's upper torso weight to stretch the spine, to neck braces and other smaller devices that work on localized areas of the spine.

### SUMMARY OF THE INVENTION

The present invention provides a spinal traction device that utilizes some of a user's body weight in performing spinal traction therapy along a substantial length of the user's spine, but which does not require the user to be suspended, and which can be used by the user alone, without the aid of another user. The device includes a body support for supporting a majority of a user's body, including the torso, and a movable leg support that moves relative to the body support and applies tensile forces to the user's spine in response to the application of leg weight to the leg support.

According to one form of the present invention, a spinal traction device includes a body support member for supporting the upper body of a user in a supine position, a leg support for supporting the legs of the user. The body support member has a head end portion, a leg end portion, and an upper support surface. A body retaining element is provided at the body support member, for engaging and retaining a portion of the user at the upper support surface. The leg support is movably coupled to the leg end portion of the body support member. The user's legs are primarily supported at a distal end portion of the leg support, which is movable from a first position in which the distal end portion of the leg support is closer to the body retaining element of the body support member, to a second position in which the distal end portion of the leg support is spaced further from the body retaining element of the body support member. The leg support is movable from the first position to the second position in response to the application of force by the legs of the user to the distal end portion of said leg support. For example, the application of force by the legs may be due to gravity (i.e., leg weight), or the application of downward force by the leg muscles and/or the body or torso muscles acting on the legs.

In one aspect, the proximal end portion of the leg support is pivotably coupled to the leg end portion of the body support member in a manner that allows the application of force by the legs to the distal end portion of the leg support, to cause the leg support to move toward the second position.

In another aspect, the proximal end portion of the leg support is coupled to the leg end portion of the body member in a manner that allows the leg support to translate relative to the body support member as it moves toward the second position under the application of force by the legs to the distal end portion of the leg support. Optionally, the leg support is coupled to the leg end portion of the body support member in a manner that allows it to both pivot and translate between the first position and the second position.

In still another aspect, a rack-and-pinion joint movably couples the leg support to the body support member, which permits the leg support to both pivot and translate between the first position and the second position.

For example, the proximal end portion of the leg support may include a pinion of the rack-and-pinion joint, the pinion having a pivot axis, while the leg end portion of the body support member may include a rack portion of the rack-and-pinion joint. Optionally, the leg end portion of the body support defines a slot for receiving a shaft disposed along the pivot axis of the pinion located at the proximal end portion of the leg support. The shaft is disposed in the slot and is permitted to translate along the slot as the pinion of the leg support pivots and translates along the rack portion.

Optionally, the pinion has an oblong shape and the slot is arcuate, to receive and guide the shaft as it traces an arcuate path induced by the oblong shape of the pinion, where the oblong pinion provides an upward component to the translating motion of the leg support. Optionally, the pinion has an at least partially-circular shape and a shaft spaced radially outwardly from the pivot axis so that the shaft traverses an arcuate path when the pinion pivots about the pivot axis, and the slot comprises an arcuate shape corresponding to the arcuate path of the shaft.

According to a further aspect, the body support member includes an upper support surface for supporting the upper body of the user in the supine position, and a lower support surface for engaging a floor or ground surface. The upper support surface of the body support member is arcuate and upwardly concave or concave-up in shape. Optionally, the upwardly concave arcuate shape of the upper support surface of the body support member defines an anti-lordotic curve. Optionally, the lower support surface of the body support member has a downwardly convex or convex-down arcuate shape that permits the spinal traction device to be reciprocated in a rocking motion on the floor or ground surface.

In still another aspect, the body support member includes at least one support leg for supporting the spinal traction device in a substantially fixed orientation relative to the floor or ground surface.

In another aspect, the body retaining element of the spinal traction device is an under-arm support extending upwardly from the body support member near the head end portion, the under-arm support for supporting the underarm of the user in the supine position on the body support member. The under-arm support supports the upper body of the user in a substantially fixed position at the body support member as the leg support is moved from the first position to the second position. Optionally, the under-arm support is repositionable and releasably securable along the body support member to accommodate users of different heights and sizes. Optionally, the body retaining element is a strap or a high-friction surface for engaging a portion of the user.

Accordingly, the spinal traction device of the present invention provides spinal therapy along a substantial length of a user's spine, including but not limited to the thoracic and lumbar regions of the spine, by using the weight of the user's legs to apply a pulling or tensile force on the spine and its associated muscles. The device can be used by a patient or user without the aid of another user. The device includes a body support member for supporting the upper body of a user in a supine position, and a leg support that is movably coupled to the body support member and arranged so that at least a distal end portion of the leg support supports the legs of the user lying on the body support member.

These and other objects, advantages, purposes, and features of the invention will become more apparent upon review of the following specification in conjunction with the drawings.

### BRIEF DESCRIPTION OF THE DRAWINGS

FIG. 1A is a side elevation of a spinal traction device in accordance with the present invention, with a user lying on the device in a supine position, and with a leg support of the device in a first or partially-retracted position;

FIG. 2A is a side elevation of the spinal traction device of FIG. 1A, in which the leg support is in a second or partially-extended position;

FIG. 2A is a perspective view of the spinal traction device, in which the leg support is at a first or partially-retracted position;

FIG. 2B is a perspective view of the spinal traction device of FIG. 2A, with the leg support in a second or partially-extended position;

FIG. 2C is a perspective view of the spinal traction device of FIG. 2A, with the leg support in a third or fully-extended position;

FIG. 3A is an enlarged perspective view of a rack-and-pinion joint of the spinal traction device of FIGS. 2A-2C, with the leg support shown in a fully-retracted position;

FIG. 3B is another perspective view of the rack-and-pinion joint, shown with the leg support in a partially-extended position;

FIG. 3C is another perspective view of the rack-and-pinion joint, with the leg support in a fully-extended position;

FIGS. 4A-C are perspective views of an inboard portion of the rack-and-pinion joint, and corresponding to the perspective views of FIGS. 3A-C;

FIG. 5 is a perspective view of the underside of the leg support of the spinal traction device of FIGS. 2A-C, including a diagrammatic representation of an adjustable calf support;

FIG. 6 is an enlarged perspective view of a portion of an under-arm support of the spinal traction device of FIGS. 2A-C;

FIG. 7 is another enlarged perspective view of a portion of the under-arm support, shown detached and spaced from the body support member;

FIG. 8 is a side elevation of another rack-and-pinion joint, having an oblong pinion;

FIG. 9 is a side elevation of another rack-and-pinion joint, having a circular pinion with an off-center mounting shaft;

FIG. 10A is a perspective view of another spinal traction device in accordance with the present invention, having a fixed leg support and being positioned in a first orientation on a support surface; and

FIG. 10B is another perspective view of the spinal traction device of FIG. 10A, being positioned in a second orientation on the support surface.

### DESCRIPTION OF THE PREFERRED EMBODIMENTS

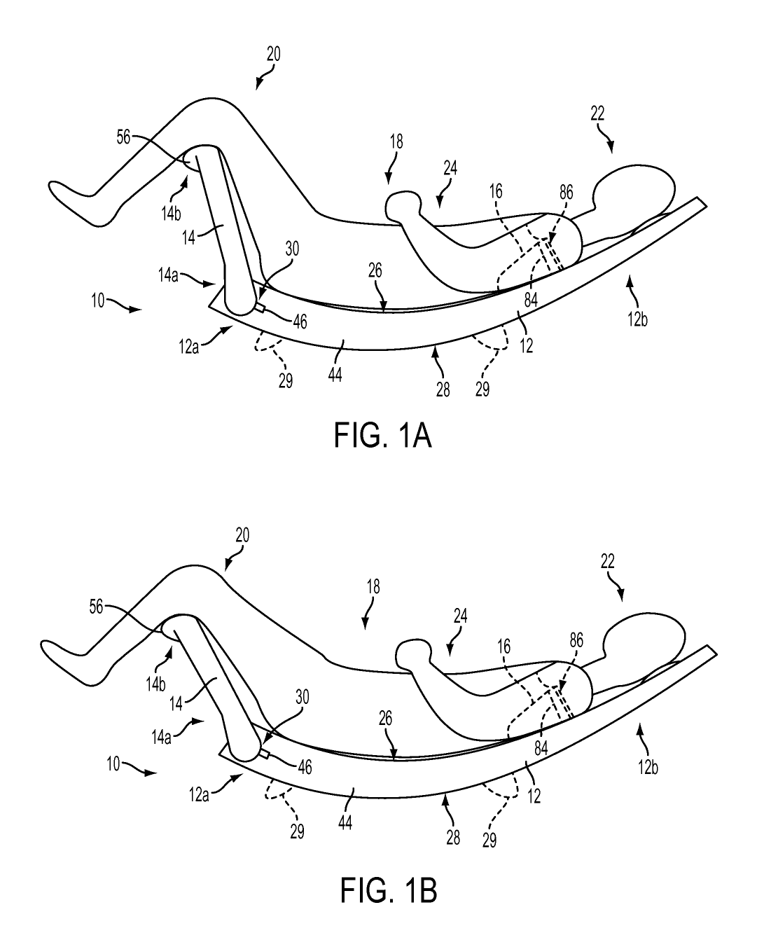

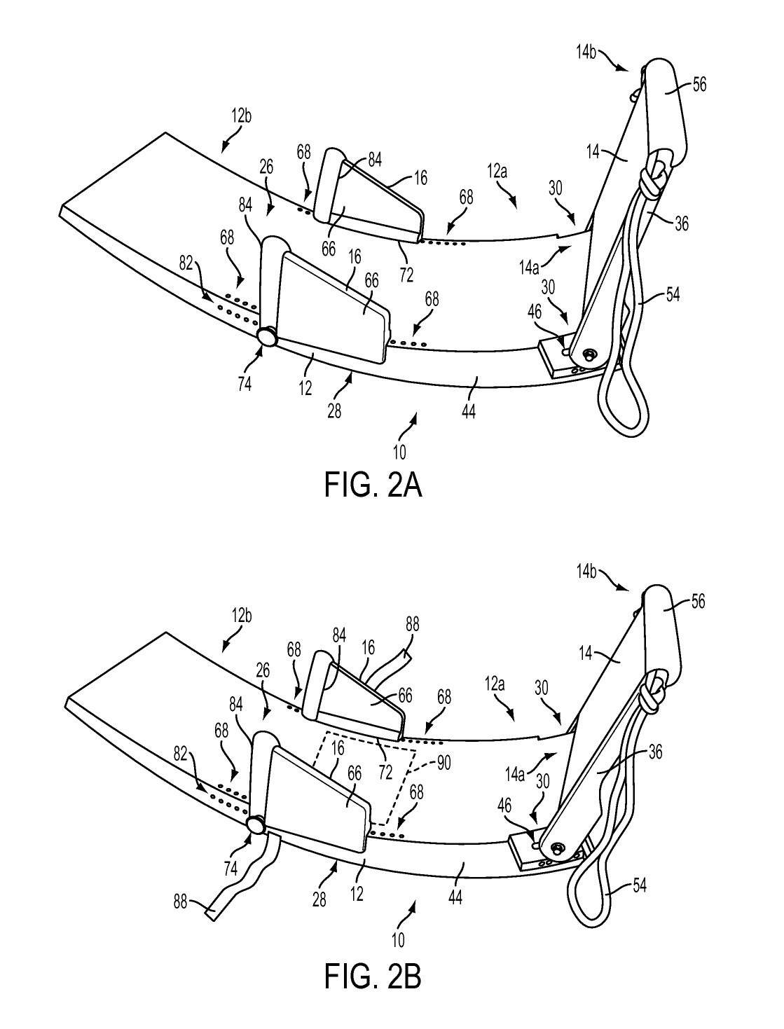

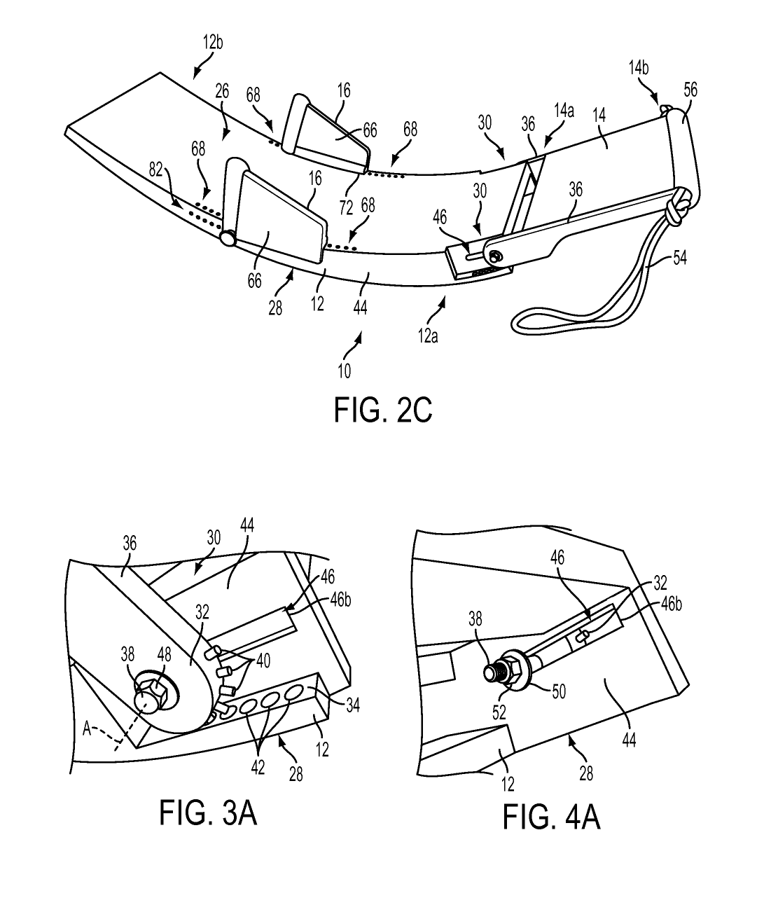

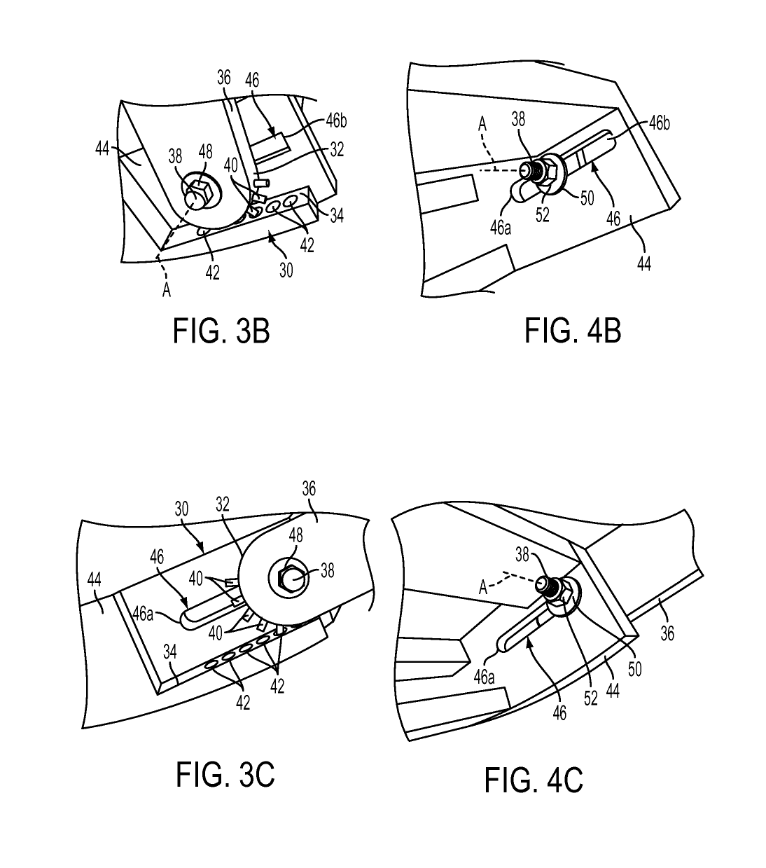

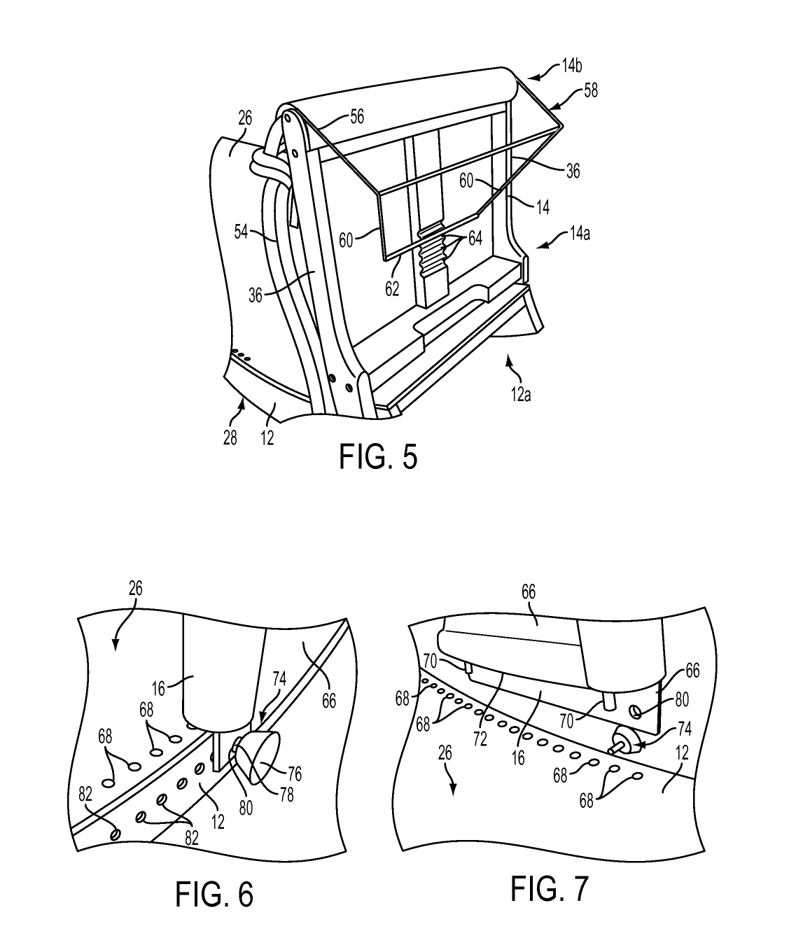

Referring now to the drawings and the illustrative embodiments depicted therein, a spinal traction device 10 includes a body support member 12, a leg support 14 movably coupled to body support member 12, and under-arm supports 16, for supporting a patient or user 18 lying in a supine position atop the device 10 (FIGS. 1A-B). Leg support 14 may be moved or repositioned to substantially any number of positions between a fully-retracted position (e.g., FIGS. 3A and 5) and a fully-extended position (FIGS. 2C and 3C) although, as will become apparent, the range of travel of leg support 14 is typically limited by the size and/or flexibility of user 18. In the first position of FIG. 1A, for example, little or no tension is applied to the user's spine, while in the second position of FIG. 1B, the user's legs 20 are permitted to drop under the weight of gravity, causing leg support 14 to move and apply a force to the backsides of the user's knees and/or to the calves.

Body support member 12 has an upwardly concave or concave-up arcuate upper surface 26 for supporting the user's torso 24 and head 22, and has an arcuate, downwardly-convex or convex-down lower surface 28 for supporting spinal traction device 10 and user 18 on a floor or other support surface. Body support member 12 includes side members 44 extending between arcuate upper surface 26 and arcuate lower surface 28 (and forming arcuate lower surface 28). Spinal traction device 10 may be permitted to rock or reciprocate with arcuate lower surface 28 of body support member 12 in engagement with a floor or other support surface, as user 18 shifts his or her weight. Optionally, one or more supports or stabilizer feet 29 (FIGS. 1A and 1B) may extend below arcuate lower surface 28 for stabilizing spinal traction device 10 to hold it in a fixed position relative to the floor or support surface on which the device is positioned (FIGS. 1A and 1B).

Leg support 14 includes a proximal end portion 14a that is movably coupled to a leg end portion 12a of body support member 12, and further includes a distal end portion 14b for supporting the legs 20 of user 18. A head end portion 12b of body support member supports at least the head 22 of user 18, while the remainder of body support member 12 generally supports the user's torso 24. Leg support 14 is movable between a first position in which distal end portion 14b is spaced a reduced distance from under-arm supports 16 (FIG. 1A), and a second position in which distal end portion 14b is spaced a greater distance from under-arm supports 16 to apply traction therapy to the user's spine (FIG. 1B). In the illustrated embodiment, leg support 14 both pivots and translates relative to body support member 12 as it moves between the first and second positions, as will be described below.

Proximal end portion 14a of leg support 14 and leg end portion 12a of body support member 12 combine to form a rack-and-pinion joint 30 in which leg support 14 forms a pinion 32, and body support member 12 forms a rack portion 34, as best shown in FIGS. 3A-C. In the illustrated embodiment, each pinion 32 is formed at a proximal end of a respective side arm 36 of leg support 14. The outer surface of pinion 32 forms a portion of a circle to-create a substantially constant-radius arc around a pivot axis A (FIGS. 3A and 3B). A shaft 38 extends through a bore (not shown) in pinion 32 along axis A, and attaches leg support 14 to body support member 12, as will be described below. Pinion 32 includes a plurality of evenly-spaced radial projections 40 that selectively engage and intermesh with rack portion 34 as leg support 14 moves relative to body support member 12.

Rack portion 34 defines a plurality of evenly spaced bores 42 arranged rectilinearly in a line (FIGS. 3A-3C), the bores 42 having respective longitudinal axes that are parallel to one another and substantially perpendicular to pivot axis A of pinion 32. Bores 42 are spaced and aligned to intermesh with radial projections 40 of pinion 32 so that proximal end portion 14a of leg support 14 is forced to translate along leg end portion 12a of body support member 12 when leg support 14 is pivoted so that pinion rotates 32 and successive projections 40 engage successive bores 42. Thus, leg support 14 is forced to pivot when its proximal end portion 14a translates along the leg end portion 12a of body support member 12. Optionally, it will be appreciated that a rack-and-pinion joint may be formed from more conventional toothed gears along the pinion and rack, rather than the radial projections 40 and bores 42 described above.

Side members 44 of body support member 12 define elongate slots 46 at leg end portion 12a of body support member 12, for receiving shafts 38 of pinions 32. In the illustrated embodiment, shafts 38 are threaded bolts having heads 48 disposed at the outboard surfaces of pinions 32, along axes A that extend through slots 46, as best shown in FIGS. 3A-3C. Shaft 38 extends through a respective elongate slot 46 and is captured in the slot by attachment of a washer 50 and a threaded nut 52 along the inboard side of each side member 44 (FIGS. 4A-4C). Washer 50 has a diameter that is greater than the width of elongate slot 46 to prevent pull-through of shaft 38, and so that washer 50 slides smoothly along an inner surface of side member 44, near slot 46, as leg support 14 pivots and translates relative to body support member 12.

When leg support 14 is in a fully-retracted position, leg support 14 is prevented from further retraction and moving beyond that position by engagement of side arms 36 with either arcuate upper surface 26 of body support member 12, or with a portion of the side members 44 (FIG. 3A). However, it will be appreciated that in the fully-retracted position, shaft 38 may contact a proximal end 46a of slot 46, to limit or prevent further movement of leg support 14. Rack-and-pinion joint 30 forces leg support 14 to pivot and translate simultaneously as it is moved between the fully-retracted position of FIGS. 3A and 4A, and the fully-extended position of FIGS. 3C and 4C, so that radial projections 40 of pinion 32 intermesh with the bores 42 in rack portion 34, and shaft 38 simultaneously slides along slot 46, from the slot's proximal end 46a toward its distal end 46b. Once leg support 14 reaches its fully-extended position (FIGS. 2C, 3C, and 4C), shafts 38 engage the respective distal 46b of slots 46, which prevents further translation of leg support 14 and, due to engagement of radial projections 40 with bores 42, also prevents further pivoting movement of the leg support beyond the fully-extended position.

It will be appreciated that the leg support may be attached to the body support member in a manner such that it only translates and does not pivot relative to the body support member. Optionally, the leg support may be pivotably attached to the body support member, such as with a simple hinge or pivot joint, so that it only pivots relative to the body support member to change the distance between the under-arm supports and the distal end of the leg support. Thus, either or both of pivoting and translating motion of the leg support, relative to the body support member, can be used to achieve traction with the device, since both types of motion can be used to position the distal end of the leg support further from the under-arm supports 16.

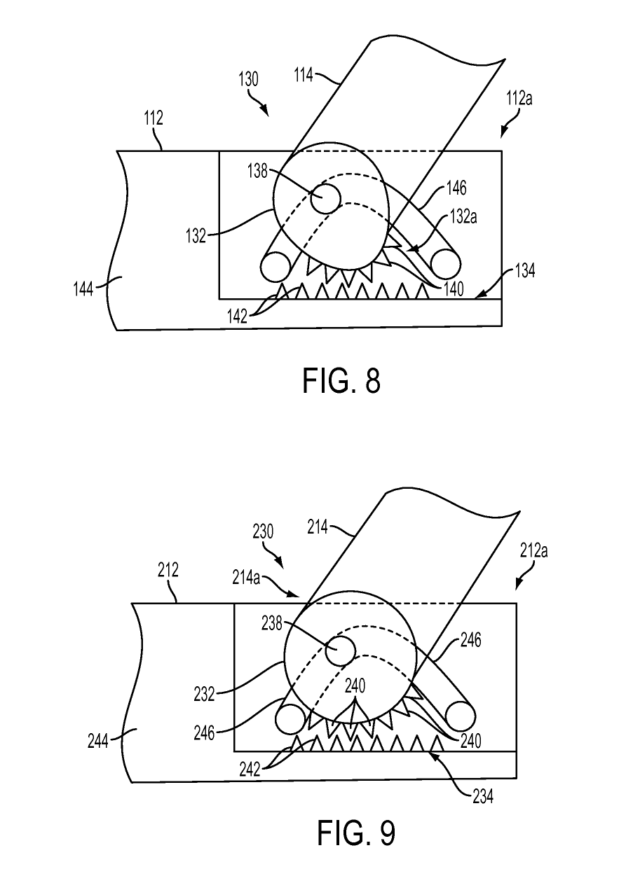

Optionally, a rack-and-pinion joint 130 including an oblong pinion 132 and a toothed rack portion 134 may be used to couple a leg support 114 to a body support member 112 (FIG. 8). Pinion 132 includes a plurality of gear teeth 140 along at least a portion of the outer surface of the pinion, which intermesh with corresponding gear teeth 142 along the rack portion 134. In the illustrated embodiment, oblong pinion 132 has a lower end portion defining a lobe 132a from which gear teeth 140 project. A shaft 138 is coupled to pinion 132 and extends through an arcuate slot 146 in a respective side member 144 of the body support member 112. Arcuate slot 146 is shaped to correspond to the path traced or followed by shaft 138, according to the shape and dimensions of lobe 132a and the position of shaft 138 in pinion 132. Shaft 138 travels with the pinion 132 as the pinion moves along the rack portion 134, and its engagement with arcuate slot 146 maintains the intermeshing engagement of gear teeth 140 with gear teeth 142 as leg support 114 pivots and translates relative to body support member 112. The oblong shape of pinion 132, and particularly the lobe 132a of pinion 132, causes leg support 114 to extend somewhat upwardly away from the leg end portion 112a of body support member 112 as force is applied to leg support 114, which can enhance the traction therapy achievable by a spinal traction device incorporating rack-and-pinion joint 130.

Another rack-and-pinion joint 230 includes a generally circular pinion 232 and a toothed rack portion 234 may be used to couple a leg support 214 to a body support member 212 (FIG. 9). Pinion 232 includes a plurality of gear teeth 240 along at least a portion of the outer surface of the pinion, which intermesh with corresponding gear teeth 242 along the rack portion 234. A shaft 238 is coupled to pinion 232 at a position that is spaced radially outwardly from the center or center of rotation of the pinion, so that shaft 238 traces or follows an arcuate path as pinion 232 rotates. An arcuate slot 246 in a respective side member 244 of the body support member 212 is shaped corresponding to the path traced or followed by shaft 238, which can be adjusted according to the position of shaft 238 in pinion 232. Shaft 238 travels with the pinion 232 as the pinion moves along the rack portion 234, and its engagement with arcuate slot 246 maintains the intermeshing engagement of gear teeth 240 with gear teeth 242 as leg support 214 pivots and translates relative to body support member 212. The motion of leg support 214 is substantially similar to that of leg support 14 with rack-and-pinion joint 30, described above, in that a proximal end portion 214a of leg support 14 will translate substantially linearly along leg end portion 212a of body support member 212 as force is applied to leg support 214. Optionally, and depending on the manner and location at which leg support 214 is coupled to pinion 232, rack-and-pinion joint 232 may provide the same or similar motion as joint 132, including an upward component to the translating motion of leg support 114.

A pair of ropes or straps 54 are provided at distal end portion 14a of leg support 14 (FIGS. 2A-2C and 5) for use as hand-holds, which can be used to aid a user as they position themselves on spinal traction device 10, or to limit the degree of traction provided by the device, as will be described below. Distal end portion 14b further includes a cushion or pad 56 to engage and support the backs of the user's knees, such as shown in FIGS. 1A and 1B. Optionally, an extended calf support 58 may extend from, or act in concert with, cushion 56, such as shown diagrammatically in FIG. 5. Calf support 58 may be adjustable to provide different angles for support of the user's calves along their lower legs, and includes support rods 60 that are connected with one another at a cross bar 62, which is selectively engageable with one of a plurality of notches 64 formed in a bottom surface of leg support 14. Movement of cross bar 62 to different notches 64 causes corresponding adjustment of the angle of support 58 with respect to the upper surface of leg support 14.

Under-arm supports 16 are positionable at a plurality of locations along respective sides of body support member 12, such as shown in FIGS. 2A-2C. In the illustrated embodiment, each under-aim support 16 includes a generally vertically-aligned plate 66 that extends above and partially below arcuate upper surface 26 to define a shoulder 72, with at least a lower portion of plate 66 extending alongside side member 44. At least a portion of each plate 66 is positioned directly above a respective side edge of arcuate upper surface 26 to define shoulder 72. Arcuate upper surface 26 defines a plurality of evenly-spaced bores 68 that are configured to receive pins 70 that extend downwardly from an underside of shoulder 72 of plate 66 (FIG. 7). Pins 70 are spaced to align with respective ones of bores 68 in upper surface 26 of body support 12, so that under-arm supports 16 can be placed and secured at an appropriate position corresponding to the body size of user 18 when pins 70 are telescoped or inserted into respective ones of bores 68.

A removable locking member 74 is used to lock under-arm supports 16 along body support member 12, and includes a handle or knob portion 76 and a locking shaft 78. Locking shaft 78 extends through a bore 80 in a lower portion of plate 66 (FIGS. 6 and 7) and into one of a plurality of bores 82 defined in side member 44 of body support member 12. Bores 82 in side members 44 are spaced correspondingly to bores 68 in upper surface 26 so that the bore 80 in plate 66 aligns with one of bores 82 when pins 70 engage respective bores 68 in upper surface 26 (FIG. 6). Optionally, each under-arm support 16 includes a pad 84 along a head end of plate 66, to act as a cushion for the underarm or armpit 86 of the user 18 lying supine on spinal traction device 10, such as shown in FIGS. 1A and 1B.

It will be appreciated that many different methods are envisioned for movably and securely coupling the under-arm supports to the body support member. For example, the under-arm supports could be movably secured to a guide track, with spring-loaded release pins or catches or a ratcheting mechanism provided for lockably and releasably engaging the guide track. The width or lateral distance between the under-arm supports could also be adjustable to accommodate a range of body sizes, such as by using telescoping rods or the like, for example.

Thus, a user can lie on the device without need for being strapped to the device, since the user is always positioned above or atop the device during its use. However, it will be appreciated that leg straps, arm or chest straps 88 (FIG. 2B), and a head cradle and/or strap may be used to further secure the user to the device, or to provide traction therapy to additional parts of the spine such as the cervical region. For example, leg straps may be applied near the user's knees to aid the user in maintaining proper positioning of the legs on the leg support; arm straps may be used in place of under-arm supports; and a head strap can be positioned over the user's forehead so that at least some tension is applied to the cervical region of the user's spine as the legs are lowered on the leg support. Optionally, the upper surface of the body support member may include a high-friction pad or surface 90 (FIG. 2B) for supporting at least a portion of the user's weight and resisting sliding movement of at least a portion of the user along the upper surface. For example, a high-friction pad could be positioned underneath the user's shoulders, to substantially prevent the user's shoulders from sliding along the upper surface of the body support member during traction therapy in which forces are applied to the user's calves or the backs of the legs by the leg support, so that those forces are transferred to the user's spine and related muscles.

To use spinal traction device 10 for spinal traction therapy, user 18 first positions the traction device on a floor or suitable support surface and then typically sits on leg end portion 12a of body support member 12 while facing leg support 14, with legs 20 straddling the leg support 14. The legs 20 are then positioned atop leg support 14, with leg support 14 positioned anywhere between a fully-extended position (FIG. 2C) and a partially-extended or in-use position (FIG. 1A). If user 18 has initially sat upon body support member 12 with legs 20 outstretched along leg support 14 in the fully-extended position of FIG. 2C, user 18 may lie back to a fully-supine position while grasping ropes 54 to draw leg support 14 upwardly (i.e. pivoting and translating leg support 14 away from its fully-extended position) until cushion 56 engages the backs of the user's knees (FIG. 1A). Optionally, if user 18 is initially seated upright on body support member 12 with the legs 20 positioned over leg support 14, the user may use ropes 54 to gently lower their upper torso and head toward the arcuate upper surface 26 of body support member 12 until they are in the fully-supine position of FIG. 1A.

Once in the supine position, with armpits 86 positioned over the pads 84 of under-arm supports 16 (FIG. 1A), the user may begin traction therapy by allowing the weight of their legs 20 to press against leg support 14 (and optionally calf support 58, if included) and move the leg support in the direction of its fully-extended position, such as shown in FIG. 1B as compared to FIG. 1A. User 18 maintains his/her legs 20 in an at least partially-bent orientation, so that cushion 56 (and optionally calf support 58) pushes against the user's calves and/or the backs of the knees as the weight of their legs 20 pushes against leg support 14. This causes the leg support to both pivot and translate (due to the action of rack-and-pinion joint 30) so that the weight of the legs 20 increases the distance between under-arm supports 56 and distal end portion 14b of leg support 14. This increasing distance applies tension to the user's spine by applying force to the backs of the user's calves or knees with cushion 56 of leg support 14, while holding the position of armpits 86 fixed at under-arm supports 16.

Spinal traction device 10 can thus be used to perform traction on a substantial length of the user's spine, including substantially the entire lumbar portion and thoracic portion (approximately to the shoulders). The curvature of the arcuate upper surface 26 of body support member 12 itself provides an anti-lordotic effect (i.e., counteracts abnormal forward curvature of the lumbar portion of the spine) and reduces spinal stenosis (i.e., counteracts the effects of narrowing of the spinal canal and compression of the spinal cord and nerves) by increasing blood flow as the spine flexes and its discs pivot or move relative to one another. The user 18 lying atop spinal traction device 10 controls the degree of traction by using muscles in the legs and torso to control the amount of leg weight that is permitted to act upon leg support 14, which directly controls or affects the change in distance between distal end portion 14b of leg support 14 and under-arm supports 16, thereby controlling tension or traction. To increase the amount of traction, the user allows more leg weight to act upon leg support 14, or can even use the leg muscles to apply additional force to leg support 14. Alternatively, the user can use the leg muscles to reduce the amount of force acting on leg support 14 to thereby reduce tension and reduce the degree of traction. Ropes 54 may also be grasped in the user's hands and used to help control or limit the degree of travel of leg support 14 such as shown in FIGS. 1A and 1B,

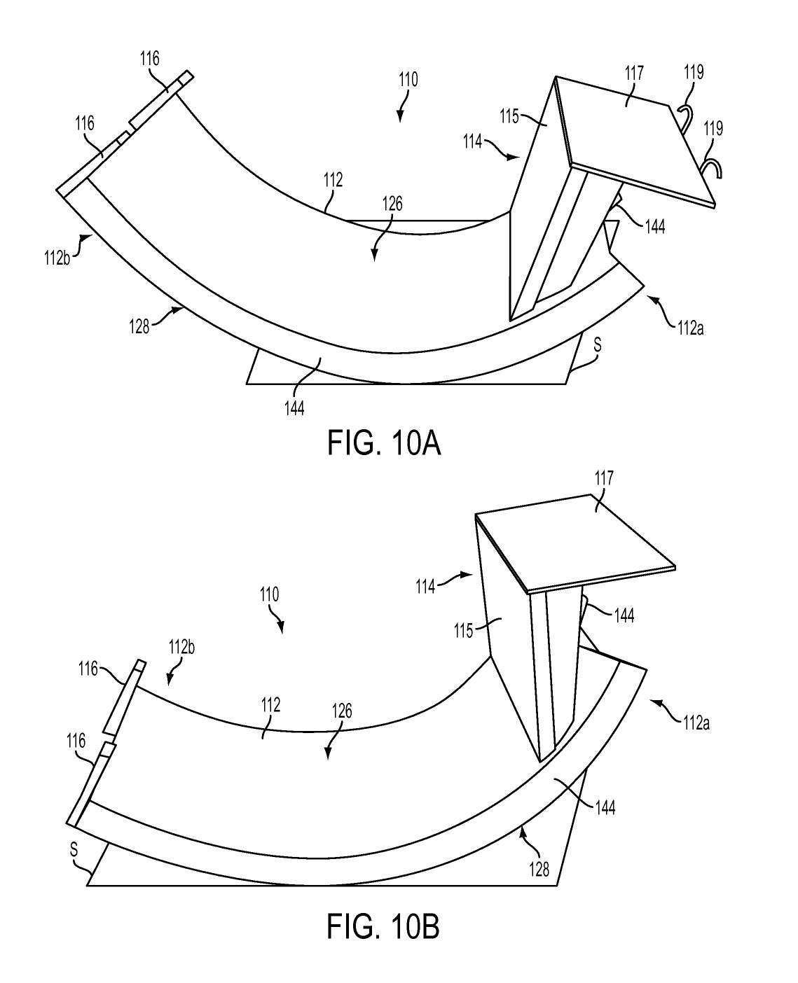

Optionally, and with reference to FIGS. 10A and 10B, an alternative spinal traction device 110 includes a body support member 112, a leg support 114 fixedly attached to body support member 112 at a leg end portion 112a, and shoulder-top supports 116 at a head end portion 112b of body support member 112. Body support member 112 includes an arcuate upper surface 126 for supporting at least the torso of a person lying thereon, and arcuate lower surfaces 128 formed by a pair of side members 144 for engagement with a support surface S. Like upper surface 26 of spinal traction device 10, upper surface 126 is capable of providing an anti-lordotic effect and reducing spinal stenosis. For reasons that will be discussed below, it may be desirable to provide a low-friction material along upper surface 126.

Leg support 114 includes a thigh or upper-leg support surface 115 extending upwardly away from upper surface 126 of body support member 112 at an angle (such as a 45-degree angle), and further includes a calf or lower-leg support surface 117 (which may be a high-friction surface) extending from thigh support surface 115 at an angle (such as a 90-degree angle). Optionally, the angles between upper surface 126 and thigh support surface 115, and between thigh support surface 115 and calf support surface 117, may be adjustable and releasably lockable. However, when spinal traction device 110 is in use, the angles and relative positioning of leg support 114 are generally held fixed. Optionally, leg straps or other leg retaining devices are provided at leg support 114, such as ankle brackets 119 along calf support surface 117 (FIG. 10A).

Shoulder-top supports 116 are provided to engage the tops of a person's shoulders in the event that the person's torso slides along upper surface 126 in the direction of head end portion 112b of body support member 112. Thus, supports 116 can prevent the person from sliding off of upper surface 126 in the direction of head end portion 112b. Shoulder-top supports 116 are laterally spaced to provide room for a person's head and/or neck to extend between the supports 116, although normally, the person's head and neck are positioned atop upper surface 126 near head end portion 112b.

To use spinal traction device 110, a person positions himself or herself atop arcuate upper surface 126 of body support member 112 with the legs positioned over leg support 114. The user's legs may be retained at leg support 114 using ankle brackets 119 or other retaining devices, if provided. The person then shifts their torso weight and/or pushes off of the support surface S with their arms (or an assistant may push against the device 110) to rock the device 110 backwards in a manner that lowers head end portion 112, and therefore the person's upper torso and head, closer to support surface S, while raising the person's legs, such as shown in FIG. 10B.

With spinal traction device 110 oriented in a tilted-back traction position as in FIG. 10B, the upper body weight of the person's upper torso, head, neck, and arms applies tension to the person's spine. The upper body weight causes the person's upper body to slide along upper surface 126 in the direction of head end portion 112b and shoulder-top supports 116 as the person's legs are held substantially fixed on lower leg support 114, thus applying tension to the spine. It will be appreciated that a low-friction material along upper surface 126 of body support member 12 will facilitate sliding motion of the person's upper body along the upper surface 126, and therefore increase the degree of traction therapy achieved when the device 110 is tilted back to the traction position. Optionally, the person positioned on the device 110 (or an assistant) may impart a rocking motion to the device, providing a cyclical traction therapy to the spine and increasing the anti-lordotic and stenosis-reducing effects of the device due to cyclical flexion of the person's spine along the arcuate upper surface 126.

Thus, the spinal traction device of the present invention can be used by a single user, with or without the aid of another, to conduct spinal traction along a substantial portion of the user's spine. The device uses the user's leg weight and/or leg and/or body or torso muscle action to apply traction to the spine, without requiring that the user strap themselves to the device, suspend themselves in an inverted position or in any other suspended position, and permits the user to control the desired degree of the traction therapy applied by the device.

Changes and modifications in the specifically described embodiments can be carried out without departing from the principles of the present invention, which is intended to be limited only by the scope of the appended claims, as interpreted according to the principles of patent law including the doctrine of equivalents.

We claim:

1. A spinal traction device comprising:
a body support member configured to support an upper body of a user in a supine position, said support member having a head end portion, a leg end portion, and an upper support surface;
a body retaining element at said upper support surface, said body retaining element configured to engage and at least partially retain a part of the user at said upper support surface;
a leg support having a proximal end portion for coupling to said body support member at said leg end portion thereof, and having a distal end portion for supporting the legs of a user, said leg support being movably coupled to said body support member; and
wherein said leg support is movable from a first position in which said distal end portion is closer to said body retaining element of said body support member, to a second position in which said distal end portion is further from said body retaining element of said body support member than when in said first position, in response to the application of force by the legs of the user to said distal end portion of said leg support.

2. The spinal traction device of claim 1, wherein said proximal end portion of said leg support is translatably coupled to said leg end portion of said body support member, whereby the application of force by the legs of the user to the distal end portion of said leg support causes said leg support to translate toward said second position.

3. The spinal traction device of claim 1, wherein said proximal end portion of said leg support is pivotably coupled to said leg end portion of said body support member, whereby the application of force by the legs of the user to the distal end portion of said leg support causes said leg support to pivot toward said second position.

4. The spinal traction device of claim 3, wherein said proximal end portion of said leg support is translatably coupled to said leg end portion of said body support member, whereby the application of force by the legs of the user to the distal end portion of said leg support causes said leg support to translate toward said second position.

5. The spinal traction device of claim 4, further comprising a rack-and-pinion joint for movably coupling said leg support to said body support member.

6. The spinal traction device of claim 5, wherein said proximal end portion of said leg support comprises a pinion of said rack-and-pinion joint, said pinion having a pivot axis, and wherein said leg end portion of said body support member comprises a rack portion of said rack-and-pinion joint.

7. The spinal traction device of claim 6, wherein said leg end portion of said body support member further comprises a slot, and said proximal end portion of said leg support comprises a shaft disposed at said pivot axis of said pinion, and wherein said shaft is disposed in said slot and is permitted to translate along said slot as said pinion of said leg support pivots and translates along said rack of said leg end portion of said body support member.

8. The spinal traction device of claim 6, wherein said pinion comprises an oblong shape and said slot comprises an arcuate shape, said pinion configured to provide an upward component to the translating motion of said leg support.

9. The spinal traction device of claim 6, wherein said pinion comprises an at least partially circular shape and a shaft spaced radially outwardly from said pivot axis so that said shaft traverses an arcuate path when said pinion pivots about said pivot axis, and wherein said slot comprises an arcuate shape corresponding to said arcuate path of said shaft.

10. The spinal traction device of claim 1, wherein said body support member comprises an upper support surface and a lower support surface, said upper support surface for supporting the upper body of the user in the supine position, said lower support surface for engaging a floor or ground surface, and wherein at least said upper support surface of said body support member comprises an upwardly concave arcuate shape.

10. The spinal traction device of claim 10, wherein said upper support surface of said body support member comprises an anti-lordotic curve.

12. The spinal traction device of claim 10, wherein said lower support surface of said body support member comprises a downwardly convex arcuate shape that permits said spinal traction device to be moved in a reciprocating or rocking motion on the floor or ground surface.

13. The spinal traction device of claim 10, wherein said lower support surface of said body support member comprises at least one support leg configured to support said spinal traction device in a substantially fixed orientation relative to the floor or ground surface.

14. The spinal traction device of claim 1, wherein said body retaining element comprises an under-arm support extending upwardly from said body support member near said head end portion thereof, and said under-arm support is configured to support the underarm of the user in the supine position to thereby support the upper body of the user in a substantially fixed position at said body support member as said leg support is moved from said first position to said second position.

15. The spinal traction device of claim 14, wherein-said under-arm support is repositionable and releasably securable along said body support member to accommodate users of varying heights and/or sizes.

16. The spinal traction device of claim 1, wherein said body retaining element comprises a high-friction surface positioned along said upper surface of said body support member.

17. The spinal traction device of claim 1, wherein said body retaining element comprises at least one strap for releasably securing a part of the user to said upper surface of said body support member.

18. The spinal traction device of claim 1, wherein the force applied by the legs of the user to the leg support for moving the leg support comprises at least one of (i) leg weight, (ii) a downward force applied by the leg muscles, and (iii) a downward force applied by the torso muscles to the legs.

19. A spinal traction device comprising:
a body support member configured to support an upper body of a user in a supine position, said support member having a head end portion, a leg end portion, an upper support surface for supporting the upper body of the user, and a lower surface for engaging a floor surface;
a leg support mounted to and extending upwardly from said leg end portion of said body support member for supporting the legs of a user;
a leg retaining element at said leg support, said leg retaining element configured to engage and at least partially retain a portion of the legs of the user at said leg support; and
wherein said body support member is tiltable from a first position in which said head end portion is further from the floor surface, to a second position in which said head end portion is closer to the floor surface so that the user's upper body weight causes the user's upper body to move along the upper support surface to thereby apply tension to the user's spine.

20. The spinal traction device of claim 19, wherein said lower surface comprises a convex arcuate surface.
